(19) United States
(12) Patent Application Publication
(10) Pub. No.: US 2026/0137615 A1
(43) Pub. Date: May 21, 2026

(54) Anhydrous Sunscreen Compositions With enhanced Ultraviolet Protection

(71) Applicant: ELC Management LLC, Melville, NY (US)

(72) Inventors: Wei Chen, Syosset, NY (US); Snehal M. Shah, Nesconset, NY (US); Victoria Brady, West Babylon, NY (US); Elana Sava, Hewlett, NY (US); Geng Li, Brooklyn, NY (US); James Lyons, Holtsville, NY (US)

(21) Appl. No.: 18/954,915

(22) Filed: Nov. 21, 2024

(51) Int. Cl. A61K 8/86; A61Q 17/04

(52) U.S. Cl. CPC A61K 8/86; A61Q 17/04; A61K 2800/26; A61K 2800/31; A61K 2800/5922

(57) ABSTRACT

Topical anhydrous sunscreen compositions that comprise glycereth-26 and avobenzone and/or glycereth-26. In addition to boosting SPF and UVA-PF values, glycereth-26 provides excellent aesthetics, sensory profiles and spreading ability, and is particularly suitable for use in anhydrous systems. Compositions of the invention may be in the form of oil/oil, oil/silicone, or silicone/oil emulsions, as well as multi-phase anhydrous systems, such as oil/silicone/oil.

### FIELD OF THE INVENTION

The present invention is in the field of topical skincare compositions for protecting the skin against the damaging effects of ultraviolet radiation. Specifically, the present invention pertains to boosting UVA and UVB protection factors of commonly used chemical UV filters.

### BACKGROUND OF THE INVENTION

Octocrylene is well-known as an effective UV filter for mitigating the effects of UVB radiation (approximately 280-320 nm), with relatively less protection from UVA (approximately 320-400 nm). In contrast, avobenzone (butyl methoxydibenzoylmethane) is well-known as an effective UV filter for mitigating the effects of UVA radiation, with relatively less protection from UVB. Together, these ingredients offer significant protection across the UV spectrum. Other chemical UV filters that are commonly used in sunscreen products include 2-ethylhexyl salicylate and homosalate. 2-Ethylhexyl salicylate (also known as octisalate or octyl salicylate) and homosalate are organic, liquid UV filters that are known to provide some absorption of UVB radiation, but they are useful as solvents for crystalline avobenzone. 2-Ethylhexyl salicylate is also known to impart water-resistance, and enhance the photostability of other UV filters in the same composition. It is not unusual for octocrylene, avobenzone, 2-Ethylhexyl salicylate and homosalate to be present in the same sunscreen formulation.

SPF (sun protection factor) is the long standing characterization of the effectiveness of a topical formulation to protect the skin against the harmful effects of sunlight. In accepted protocols, SPF is the ratio of the dose of UVB radiation that is required to burn the skin when it is protected with a sunscreen composition, divided by the dose of UVB radiation required to burn unprotected skin. However, SPF is specific to UVB radiation. As the importance of protecting the skin against UVA radiation became apparent (95% of UV radiation from sunlight is UVA), a new protection factor specific to UVA radiation was developed. Various means of measuring UVA-PF exist, but like SPF, they are based on the ratio of the amount of UVA radiation required to elicit a response in protected skin to the amount of UVA radiation required to elicit a response in unprotected skin. For both SPF and UVA-PF, a greater value indicates more protection.

In the effort to develop sunscreen products with higher and higher SPF and UVA-PF values, simply increasing the concentration of UV filters is problematic. For example, increasing the amount of UV filter in a topical formulation may have degrading effects on the aesthetics of the product, resulting in an ashen or pasty appearance. Also, there may be some breakdown of UV filters in formulation. If this happens, sensitive skin may react to one or more of the breakdown products. For this reason, stabilizing agents may be added to the formulation to stabilize the UV filters. However, if the concentration of UV filter is increased (to boost SPF or UVA-PF), then the level of stabilizing agent would also need to be increased, to avoid irritating reactions. It would be better if the SPF and UVA-PF values of sunscreen formulations could be increased without increasing the concentration of UV filters.

SPF and UVA-PF boosters (chemical species that increase the effectiveness of UV filters) are known. However, boosting UV protection factors in anhydrous sunscreen compositions has been problematic. Not only must the boosting agent be dispersible in the anhydrous environment without diminishing the stability and other qualities of the formulation, but ideally it should boost the protection factor across the whole UV spectrum. Meeting all of these requirements has been difficult. SPF boosters that have been used in anhydrous sunscreen formulations (e.g. waxes, polymer particles, silica particles, and film formers) have negative impact on sensory profiles, esthetics or spreading ability. Moreover, there are only few UVA-PF boosters on the market that do not satisfy the needs of all types of sunscreen formulations. As such, there remains a need for new SPF and UVA-PF boosters for use in anhydrous sunscreen formulations.

Glycereth-26 is a polyethylene glycol ether of glycerin. More generally, glycerin ethoxylates have the following structure:

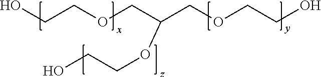

wherein x+y+z is the average ethoxylation value. So, for glycereth-26, x+y+z=26. Reported uses of glycereth-26 in personal care products include as a skin conditioning agent, humectant, dispersant, solubilizer, foam modifier, and viscosity decreasing agent. Glycereth-26 (CAS no. 31694-55-0) is liquid at room temperature, and has a molecular weight of 1237.47 g/mol. Glycereth-26 is soluble in water and alcohol, but not mineral or vegetable oils. Its use in emulsion systems has been reported. Glycereth-26 is commercially available as Liponic™ EG-1, for example, from Vantage™ Specialty Chemicals.

U.S. Pat. Nos. 8,372,382 and 8,603,508 disclose oil-in-water emulsions that comprises less than 50% water, and a blend of specific non-ionic emulsifiers and emulsion stabilizers. Also included is a specific combination humectants (glycereth-26, glycerin, betaine, panthenol, and allantoin), and one or more UV absorbing agents, particularly homosalate, octisalate, oxybenzone, avobenzone, octocrylene, and styrene/acrylates copolymer. The compositions are reported to have an SPF of at least 30.

U.S. Pat. No. 8,795,696 discloses aqueous, foamable sunscreen compositions comprising one or more pigments, TiO₂ and/or ZnO particles (at least 5% of which have a particle size greater than 100 nm), and 0.01%-30%. Optionally, the compositions may comprise butyl octyl salicylate and/or glycereth-26, functioning as an emulsion stabilizers and/or skin conditioning agents.

U.S. Pat. No. 9,333,159 discloses aqueous or anhydrous sunscreen compositions that comprise 1%-20% of mineral and/or organic UV filters (virtually any species; see columns 30-31); and 0.01%-20% moisturizer/skin conditioner agents (which may include glycereth-26 from a list of approximately 300 agents (see columns 41-43).

U.S. Pat. No. 9,937,119 discloses compositions for treating a wide array of skin conditions comprising any chemical or physical sunblock agent; one or more moisturizers (may be glycereth-26, but glycereth-26 is one of approximately 300 hundred agents listed). Example formulations 1 and 2 show an aqueous composition comprising homosalate, octisalate, oxybenzone, avobenzone, octocrylene, and styrene/acrylates copolymer (total about 21%), in combination with 4% glycereth-26. It is said that compositions of the invention can be incorporated into all types of vehicles, which may include anhydrous bases such as lipsticks and powders, but no examples are provided. Also, it should be noted that just because the base vehicle is anhydrous does not mean that the total formulation is anhydrous.

US2023/0248634 discloses aqueous compositions that enhance UV-blocking efficiency of UV blocking agents through the application of heat. The compositions comprise (1) a water phase that has 0.01%-10% of self-emulsifying polymer, and 0.1%-50% of a polyol having an IOB value less than 5 (Glycereth-26 is one of sixteen listed in Table 1); (2) an oil phase that has 0.5%-30% of organic and/or inorganic UV filter. UV blocking efficiency is said to be improved by heating the composition.

None of the foregoing references disclose or suggest an anhydrous topical sunscreen formulation comprising octocrylene and/or avobenzone and glycereth-26 as an SPF and UVA-PF boosting agent.

### OBJECTS OF THE INVENTION

A main object of the present invention is to boost the sun protection factors (SPF and UVA-PF) of commonly used UV filters.

Another object of the invention is to enhance the SPF values of topical compositions that comprise avobenzone and/or octocrylene.

Another object of the invention is to enhance the SPF values of anhydrous compositions that comprise avobenzone and/or octocrylene.

### SUMMARY OF THE INVENTION

The foregoing objectives are met by topical compositions that comprise glycereth-26 and avobenzone, glycereth-26 and octocrylene, or, preferably, glycereth-26, avobenzone, and octocrylene. Glycereth-26 is disclosed herein as an effective SPF and UVA booster that is preferential for avobenzone and octocrylene, as opposed to other commonly used UV filters. In addition to increasing SPF and UVA-PF values, glycereth-26 provides excellent aesthetics, sensory profiles and spreading ability, and is particularly suitable for use in anhydrous systems. Glycereth-26 in concentrations ranging from 1%-30% by weight of the total composition have been found to be effective. Compositions of the invention may be in the form of oil/oil, oil/silicone, or silicone/oil emulsions, as well as multi-phase anhydrous systems (e.g. oil/silicone/oil).

### DETAILED DESCRIPTION OF THE INVENTION

As used herein, the terms “sunscreen”, “sunscreen composition”, “sunscreen formulation,” and “sunscreen product” are synonymous. The essential active ingredient in a sunscreen product is a “UV filter”.

As used herein, term “comprises” means that a recitation of elements is not necessarily limited to those explicitly recited.

Composition of the present invention are topically applied to the skin for the purpose of preventing and/or ameliorating the harmful effects of ultraviolet (UV) radiation. Compositions of the invention comprise a cosmetically acceptable base, glycereth-26, and avobenzone and/or octocrylene. In some preferred embodiments of the invention, the composition is anhydrous. Compositions may comprise UV filters in addition to octocrylene and avobenzone, such as those typically used in topical skin care formulations.

To demonstrate the effects of glycereth-26 in the present context, the following anhydrous compositions (Test Samples 1-14) were prepared, and SPF and UVA-PF values were obtained following the COLIPA 2011 protocol. Spectrophotometry was performed using Labsphere UV-2000S Ultraviolet Transmittance Analyzer. Phenyl trimethicone or C12-15 alkyl benzoate were used as a cosmetically acceptable base. The United States Food and Drug Administration (21 CFR. Vol. 5, Part 352 section 10, subpart B) defines as safe and effective the use of octocrylene up to 10%, avobenzone up to 3%, octyl salicylate up to 5%, and homosalate up to 15%. In the test samples 1-14, the maximum amounts octocrylene (10%) and avobenzone (3%) were employed.

TABLE 1A
Effect of Glycereth-26 on Octocrylene UV absorbance

| Test | | | phenyl | | |
|---|---|---|---|---|---|
| Sample | glycereth- | | tri- | SPF | UVA-PF |
| no. | 26 | octocrylene | methicone | Value | value |
| 1 | 0% | 10% | 90% | 12.66 | 1.74 |
| 2 | 1% | 10% | 89% | 14.45 | 1.76 |
| 3 | 5% | 10% | 85% | 17.39 | 1.83 |
| 4 | 10% | 10% | 80% | 20.39 | 1.84 |
| 5 | 20% | 10% | 70% | 28.34 | 2.04 |
| 6 | 30% | 10% | 60% | 27.22 | 1.98 |
| control | 30% | 0% | 70% | not | not |
| A | | | | meaning- | meaning- |
| (base) | | | | ful | ful |

TABLE 1B
Percent Increase in SPF and UVA-PF
(Glycereth-26 + Octocrylene)

| Test | | % increase in | % increase in |
|---|---|---|---|
| Sample no. | glycereth-26 | SPF | UVA-PF |
| 2 | 1% | 14.1 | 1.1 |
| 3 | 5% | 37.4 | 5.2 |
| 4 | 10% | 61.1 | 5.7 |
| 5 | 20% | 123.9 | 17.2 |
| 6 | 30% | 115.0 | 13.8 |

Referring to Table 1B, the results show that SPF values vary nearly directly with the concentration of glycereth-26 (there seems to be a leveling off between 20% and 30% of glycereth-26). Even with just 1% glycereth-26, the percent increase in SPF is significant (14.1%). The SPF values more than doubled with glycereth-26 at 20% to 30%. Given that octocrylene has little innate potential to absorb UVA, it is not surprising that the results were less significant. However, a positive effect was still seen, which varies almost directly with concentration of glycereth-26. Results of control sample A (30% glycereth-26+base) were not meaningful, indicating that glycereth-30, alone, provides no UV protection.

TABLE 2A
Effect of Glycereth-26 on Avobenzone UV absorbance

| Test | | | C12-15 | | |
|---|---|---|---|---|---|
| Sample | glycereth- | | alkyl | SPF | UVA-PF |
| no. | 26 | avobenzone | benzoate | Value | value |
| 7 | 0% | 3% | 97% | 3.15 | 10.40 |
| 8 | 1% | 3% | 96% | 4.87 | 23.23 |
| 9 | 5% | 3% | 92% | 4.25 | 20.53 |
| 10 | 10% | 3% | 87% | 4.02 | 19.54 |
| 11 | 20% | 3% | 77% | 5.13 | 30.11 |
| 12 | 30% | 3% | 67% | 4.61 | 26.15 |
| control | 30% | 0% | 70% | not | not |
| B | | | | meaning- | meaning- |
| (base) | | | | ful | ful |

TABLE 2B
Percent Increase in SPF and UVA-PF
(Glycereth-26 + Avobenzone)

| Test | | % increase in | % increase in |
|---|---|---|---|
| Sample no. | glycereth-26 | SPF | UVA-PF |
| 8 | 1% | 54.6 | 123.4 |
| 9 | 5% | 34.9 | 97.4 |
| 10 | 10% | 27.6 | 87.9 |
| 11 | 20% | 62.9 | 189.5 |
| 12 | 30% | 46.3 | 151.4 |

Referring to Table 2B, the results show that UVA-PF values are significantly affected by the concentration of glycereth-26. The effect does not vary directly with concentration of glycereth-26, but the increase over the control (avobenzone alone) is consistent and large. Even with just 1% glycereth-26, the percent increase in UVA-PF is significant (123.4%). The UVA-PF values more than doubled or tripled with glycereth-26 at 20% to 30%. Given that avobenzone has relatively little ability to absorb UVB, it is not surprising that the results were not as spectacular. However, a significant positive effect of glycereth-26 on the ability of avobenzone to absorb UVB radiation was still seen. Results of control sample B (30% glycereth-26+base) were not meaningful, indicating that glycereth-30, alone, provides no UV protection.

TABLE 3
Effect of Glycereth-26 on Octocrylene + Avobenzone UV absorbance

| Test Sample | glycereth- | | | phenyl | SPF | UVA-PF |
|---|---|---|---|---|---|---|
| no. | 26 | octocrylene | avobenzone | trimethicone | Value | value |
| 13 | 0% | 10% | 3% | 87% | 16.50 | 12.44 |
| 14 | 10% | 10% | 3% | 77% | 42.24 | 23.72 |

As Table 3 shows, the effect of glycereth-26 on the combination of octocrylene and avobenzone was very significant. When 10%, glycereth-26 was added to the combination of octocrylene and avobenzone, the results were a 156.0% increase in SPF value, and a 90.7% increase in UVA-PF value.

Viewed another way, with no glycereth-26 present, adding 3% avobenzone to 10% octocrylene boosted the SPF by 30.0% (12.66 to 16.50). But when 10% glycereth-26 is also present, then the addition of avobenzone boosted the SPF by 107% (20.39 to 42.24). Likewise, with no glycereth-26 present, adding 3% avobenzone to 10% octocrylene boosted the UVA-PF by 615% (1.74 to 12.44). But when 10% glycereth-26 is also present, then the addition of avobenzone boosted the UVA-PF by 1,189% (1.84 to 23.72).

TABLE 4
Effect of Glycereth-26 on Homosalate OR Octisalate UV absorbance

| Test Sample | glycereth- | | | phenyl | SPF | UVA-PF |
|---|---|---|---|---|---|---|
| no. | 26 | homosalate | octisalate | trimethicone | Value | value |
| 15 | 0% | 10% | | 90% | 4.02 | 0.93 |
| 16 | 10% | 10% | | 80% | 5.34 | 0.94 |
| 17 | 0% | | 5% | 95% | 3.46 | 0.92 |
| 18 | 10% | | 5% | 85% | 3.70 | 0.92 |

Referring to Table 4, when combined with 10% glycereth-26, the ability of homosalate to absorb UVB increased by 32.8%. This is significant, but well less than the 61.1% increase experienced by octocrylene. The UVA absorbance of homosalate was virtually unaffected by glycereth-26 (1.1% increase in UVA-PF value), compared to a 5.7% and 87.9% increase for octocrylene and avobenzone, respectively.

When combined with 10% glycereth-26, the ability of octisalate to absorb UVB increased by 6.9%. This is well less than the 61.1% increase experienced by octocrylene, and 27.6% increase experienced by avobenzone. The UVA absorbance of octisalate was unaffected by glycereth-26 (0% increase in UVA-PF value), compared to a 5.7% and 87.9% increase for octocrylene and avobenzone, respectively.

Overall, the results show that glycereth-26 is preferential for octocrylene and avobenzone as compared to other sunscreen product ingredients, homosalate and octisalate. Based on the data above, glycereth-26 can be used in compositions of the invention at concentrations of 1%-30% by weight of the total composition. In addition to having a beneficial effect across the UV spectrum by increasing SPF and UVA-PF values, glycereth-26 provides excellent aesthetics, sensory profiles and spreading ability, and is particularly suitable for use in anhydrous systems.

In general, water, being polar, may interfere with the ability of glycereth-26 to boost the UV absorbing effectiveness of octocrylene and/or avobenzone. Therefore, preferred compositions of the invention are anhydrous, where, in the present context, “anhydrous” means less than 5% water by weight of the total composition. More preferably compositions of the invention comprise less than 1% water, even more preferably less than 0.1% water, and still more preferably less than 0.01% water by weight of the total composition. Most preferably compositions of the invention comprise 0.0% water by weight of the total composition.

Compositions of the invention may comprise UV filters other than octocrylene and avobenzone, including titanium dioxide and zinc oxide, as well as one or more of organic filters selected from the following: benzophenone-3 (oxybenzone), 4-methylbenzylidene camphor (enzacamene), benzophenone-4 (sulisobenzone), bis-ethylhexyloxyphenol methoxyphenyl triazine (bemotrizinol), diethylamino hydroxybenzoyl hexyl benzoate, diethylhexyl butamido triazone, disodium phenyl dibenzimidazole tetrasulfonate, drometrizole trisiloxane, ethylhexyl dimethyl PABA (padimate O), ethylhexyl methoxycinnamate (octinoxate), ethylhexyl salicylate (octisalate), ethylhexyl triazone, homosalate, isoamyl p-methoxycinnamate (amiloxate), isopropyl methoxycinnamate, menthyl anthranilate (meradimate), methylene bis-benzotriazolyl tetramethylbutylphenol (bisoctrizole), octocrylene, PABA (aminobenzoic acid), phenylbenzimidazole sulfonic acid (ensulizole), terephthalylidene dicamphor sulfonic acid, and mixtures thereof. Those that can are dispersible in an anhydrous system are preferred.

In compositions of the invention, the total amount of all UV filters, including octocrylene and avobenzone, may be typical for the art of sunscreen formulation. For example, the total amount of all UV filters, including octocrylene and avobenzone may range from about 0.001% to about 30%, for example, from about 0.01% to about 25%, for example, from about 0.1%-20%, for example, from about 1% to about 10% by weight of the composition. Compositions of the invention may be formulated to have a certain SPF and/or UVA-PF values ranging from about 1 to about 100, preferably from about 10 to about 50.

Optionally, compositions of the invention may comprise any ingredient that is typically used in topical skin care formulations, especially those used in anhydrous sunscreen compositions. Ingredients may be used at levels that are typical for the application as long as they do not significantly interfere with the ability of glycereth-26 to increase the UV absorbing efficiency of octocrylene and/or avobenzone. Compositions of the invention may comprise glycerin ethoxylates other than glycereth-26, such as glycereth-3, glycereth-7, glycereth-8, glycereth-12, glycereth-18, glycereth-20, glycereth-26, and glycereth-31.

Compositions of the invention may be in the form of a dispersion, suspension, solution or emulsion or some other form. Preferred emulsions are oil-in-oil, oil-in-silicone, or silicone-in-oil emulsions, as well as multi-phase anhydrous systems, such as oil/silicone/oil. One or more emulsifiers or surfactants may be used as needed, as long as they do not significantly interfere with the ability of glycereth-26 to increase the UV absorbing efficiency of octocrylene and/or avobenzone. Sunscreen compositions of the invention may be in the form of creams, lotion, gels, ointments, pastes, oils, butters, sticks, and sprays. The principles of the invention may be incorporated into wipes and towelettes. The principles of the invention may be incorporated into non-sunscreen-type products, such as powders, body washes, shampoos and color cosmetics, such as makeup foundations.

Following are non-limiting examples of anhydrous sunscreen compositions according to the present invention.

| | |
|---|---|
| Avobenzone | 3% |
| Octocrylene | 10% |
| Homosalate | 5% |
| Octisalate | 5% |
| BHT | 0.1% |
| Capryl Methicone | 16.7% |
| Dimethicone | 58.5% |
| Glycereth-26 | 1.5% |
| Vitamin E | 0.2% |

| | |
|---|---|
| Avobenzone | 3% |
| Octocrylene | 10% |
| Homosalate | 5% |
| Octisalate | 5% |
| BHT | 0.1% |
| C12-15 Alkyl Benzoate | 11% |
| Silica | 2% |
| Diisopropyl Adipate | 60.7% |
| Glycereth-26 | 3% |
| Vitamin E | 0.2% |

| | |
|---|---|
| Avobenzone | 3% |
| Octocrylene | 10% |
| Homosalate | 5% |
| Octisalate | 5% |
| BHT | 0.1% |
| Phenyl Methicone | 11% |
| Dimethicone | 57.7% |
| Polysilicone-11 | 2% |
| Dimethicone/vinyl dimethicone crosspolymer | 1% |
| Glycereth-26 | 5% |
| Vitamin E | 0.2% |

Compositions are intended to be applied to skin before extended exposure to sunlight. Depending on the exact form, compositions according to the present invention may be sprayed on the skin or applied to the skin by hand. Application should be according to a recommended dosage per unit area of skin (for example, about 2 mg of sunscreen product per cm² of skin). Compositions according to the invention should be reapplied as necessary, according to a recommended usage (for example, every two hours).

Every document cited herein is hereby incorporated herein, by reference, in its entirety. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An anhydrous topical sunscreen composition for preventing and/or ameliorating the harmful effects of ultraviolet radiation on skin, comprising:
a cosmetically acceptable base;
1% to 30% of glycereth-26 by weight of the total composition;
and one or more of avobenzone and octocrylene.

2. The anhydrous topical sunscreen composition of claim 1 wherein the total concentration of one or more of avobenzone and octocrylene is about 0.001% to about 30%.

3. The anhydrous topical sunscreen composition of claim 1 wherein the composition has an SPF value of about 10 to about 100.

4. The anhydrous topical sunscreen composition of claim 1 wherein the composition has a UVA-PF value of about 10 to about 50.

5. The anhydrous topical sunscreen composition of claim 1 wherein the composition is in the form of a dispersion, suspension, solution, emulsion, cream, lotion, gel, ointment, paste, oil, butter, stick, and sprays.

6. The anhydrous topical sunscreen composition of claim 5 wherein the composition is an oil-in-oil, oil-in-silicone, silicone-in-oil, an oil/silicone/oil emulsion.

7. The anhydrous topical sunscreen composition of claim 6 further comprising one or more emulsifiers or surfactants.
